(19) United States
(12) Patent Application Publication
(10) Pub. No.: US 2008/0206819 A1
(43) Pub. Date: Aug. 28, 2008

(54) Intensified Perfusion Production Method

(76) Inventors: Mary Tsao, South San Francisco, CA (US); Irene Shackel, South San Francisco, CA (US); Jennie P. Mather, South San Francisco, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/842,716

(22) Filed: Aug. 21, 2007

Related U.S. Application Data
(60) Provisional application No. 60/838,865, filed on Aug. 21, 2006.
(60) Provisional application No. 60/838,866, filed on Aug. 21, 2006.

(51) Int. Cl. C12P 21/00; C12N 5/02

(52) U.S. Cl. 435/70.3; 435/70.1; 435/383; 435/394; 435/404

(57) ABSTRACT

The invention comprises a process for producing a protein of interest in a perfusion system using induction agents without a substantial loss of cell viability. The invention also comprises methods of growing cells in a perfusion system using induction agents without a substantial loss of cell viability.

This application claims priority to U.S. provisional applications 60/838,866 and 60/838,865, both filed on Aug. 21, 2006, which are herein incorporated by reference in their entirety for all purposes.

### FIELD OF THE INVENTION

The invention is in the field of cell culture. Particularly the invention relates to methods of growing cells in a perfusion cell culture with induction agents without substantial loss of cell viability.

### BACKGROUND

One goal of recombinant protein production is the optimization of culture conditions to obtain the greatest possible productivity. Even incremental increases in productivity can be economically significant. Because many commercially important proteins are recombinantly produced in cells grown in culture there is a need to produce these proteins in an efficient and cost effective manner. The conditions under which cells are grown will have an impact on how economically protein can be produced. Cell culture conditions usually favor a reduction of cell viability over time, thus reducing efficiency and overall productivity.

There are four basic methods for culturing animal cells for manufacturing or production of protein products: Batch culture, Fed batch, Continuous and Perfusion Culture. Each method has its advantages and disadvantages. Batch culture is the earliest form of culture. It is carried out by placing the cells to be cultured into a fixed volume of culture medium and allowing the cells to grow. Cell numbers increase, usually exponentially, until a maximum is reached, after which growth becomes arrested and the cells die. This may be explained by either the exhaustion of a nutrient or accumulation of an inhibitor of growth or some other cause. To recover product, cells are removed from the medium either when the cells have died or at an earlier, predetermined point.

Fed Batch culture is a variation on ordinary batch culture and involves the addition of a nutrient feed to the batch. Cells are cultured in a medium in a fixed volume. Before the maximum cell concentration is reached, specific supplementary nutrients are added to the culture. The volume of the feed is minimal compared to the volume of the culture. Fed batch culture typically proceeds in a substantially fixed volume, for a fixed duration, and with a single harvest either when the cells have died or at an earlier, predetermined point.

Batch and Fed Batch method can produce significant amounts of protein. However, limitations of these methods include a reduction in cell viability over time. An average production run for these methods is approximately 15 to 20 days. Also, before each production run, the cells must be grown and prepared for inoculation into the final bioreactor. The process for preparing the cell for inoculation is time consuming and expensive. Another disadvantage is the large equipment and materials start up costs required for such a system.

In a Continuous culture, the cells are initially grown in a fixed volume of medium. To avoid the onset of the decline phase, fresh medium is pumped into the bioreactor before maximum cell concentration is reached. The spent media, containing a proportion of the cells, is continuously removed from the bioreactor to maintain a constant volume. The process also removes the desired product, which can be continuously harvested, and provides a continuous supply of nutrients, which allows the cells to be maintained in an exponentially growing state. Theoretically, the process can be operated indefinitely. Continuous culture is characterized by a continuous increase in culture volume, an increase and dilution of the desired product, and continuous maintenance of an exponentially growing culture. There is no death or decline phase.

Perfusion Culture is similar to continuous culture except that, when the medium is pumped out of the reactor, cells are not removed. As with a continuous culture, perfusion culture is an increasing-volume system with continuous harvest that theoretically can continue indefinitely. Because of the cost associated with growing the required cell population, the ability to extend the life of a fermentation process has tremendous potential for increasing productivity and minimizing costs. Ideally, once a high biomass within the bioreactor has been achieved, it would be desirable to continue the fermentation process indefinitely by continuously harvesting material from the bioreactor and replacing it with fresh nutrients.

However, traditional continuous and perfusion methods are not feasible at large scale because they require large volumes of media and have low volumetric productivity. The large volumes of media also present serious downstream purification complications and problems. In order to produce significant amounts of product, the media requirements and resulting downstream complication would make the continuous and perfusion methods impracticable. These methods would be more efficient if cells could produce more product per cell, thus increasing the volumetric productivity.

Currently, one method widely used to increase production in cells is to introduce induction agents to the culture media. Induction agents increase protein production from cells. One such induction agent is sodium butyrate. Although the exact mechanism by which sodium butyrate increases protein biosynthesis is unknown, several theories exist, including, that sodium butyrate enhances protein biosynthesis in various mammalian cells by increasing in the rate of transcription (Arts et al., (1995) Biochem. J., 310, 171 to 176; Oh et al. (1993) Biotechnol. Bioeng., 42, 601 to 610). The mechanism is likely to be associated with the increased acetylation of histones following inhibition of the enzyme acetylase (Dorner et al. (1989) J. Biol. Chem., 264, 20602 to 20607; Riggs et al (1977) Nature, 268, 462). These induction agents will induce the cell to produce more desired product. However, the trade-off with using sodium butyrate in cell culture is that cell viability is significantly compromised.

For example, in Kim et al., (2004), Biotechnol. Prog., 20, 1788 to 1796, the authors reported using sodium butyrate to increase protein production in recombinant CHO cells in a perfusion culture. The results of this study indicated that there was an increase in production of the recombinant protein but that cell viability was substantially reduced. In most cases at the end of the production run, cell viability was less than 45%. In Wang et al., (2002) Biotechnol. Bioeng., 77, 194 to 203, when the authors added sodium butyrate to recombinant CHO cells in a fluidized bed bioreactor, the cells stopped growing and cell concentration declined.

Although use of an induction agent can substantially increase volumetric productivity, the length of the production period is significantly limited by its impact on cell viability. A typical production period using an induction agent is between 12 to 15 days. Thus, the use of induction agents in a typical continuous or perfusion method would be counterproductive since such methods are designed to maintain cell viability over a longer time. Because many commercially important proteins are recombinantly produced by cells grown in culture, there is a need for increased cell productivity and more efficient production runs.

### SUMMARY OF THE INVENTION

The inventors have solved the problem related to cell viability and the use of induction agents in cell culture. The inventors have discovered novel processes and methods for producing proteins and/or polypeptides using induction agents in a perfusion culture by maintaining both high cell viability and high cell density over a production period.

Thus, the invention comprises a process for producing a protein of interest in a perfusion system, comprising culturing a cell line that expresses said protein of interest in media comprising an effective amount of an induction agent, whereby cell viability does not substantially decrease and production of said protein of interest is increased relative to cells grown without said induction agent. In one embodiment, at least about 80% to about 95% cell viability is maintained for at least 5 days in the presence of said induction agent. In another embodiment at least about 80% to about 95% cell viability is maintained for at least 10 days in the presence of said induction agent. In another embodiment at least about 80% to about 95% cell viability is maintained for at least 12 days in the presence of said induction agent. In yet another embodiment at least about 80% to about 95% cell viability is maintained about 15, about 20, about 25, or about 30 days or longer.

In another embodiment, a biomass of at least about 4 million to about 60 million viable cells per milliliter is achieved in the presence of said induction agent for at least 5 days. In another embodiment, said induction agent is sodium butyrate. In another embodiment, the concentration of sodium butyrate is increased to a final concentration of about 0.5 mM to about 2.5 mM over a period of at least 2 days.

The invention also comprises a method of culturing a cell line that expresses a protein of interest in a perfusion system, comprising culturing said cell line in media comprising an effective amount of an induction agent, whereby cell viability does not substantially decrease and production of said protein of interest is increased relative to cells grown without said induction agent. In one embodiment, at least about 80% to about 95% cell viability is maintained for at least 5 days in the presence of said induction agent. In another embodiment, said induction agent is sodium butyrate.

The invention also comprises a method of culturing a cell line that expresses a protein of interest in a perfusion system utilizing a pre-sterilized disposable bioreactor, comprising culturing said cell line in media comprising an effective amount of an induction agent wherein cell viability does not substantially decrease and production of said protein of interest is increased relative to cells grown without said induction agent and wherein said pre-sterilized disposable bioreactor is partially filled with a gas comprising oxygen and said pre-sterilized disposable bioreactor is agitated thereby agitating the liquid media in the pre-sterilized disposable bioreactor. In one embodiment, a biomass of at least about 4 million to about 60 million per milliliter viable cells is achieved in the presence of said induction agent for at least 5 days. In another embodiment, said perfusion system comprises a filter wherein said filter concentrates said protein of interest in said pre-sterilized cell culture bioreactor.

The invention also comprises a perfusion system comprising, a cell line that expresses a protein of interest and culture media, wherein said culture media comprises an induction agent in sufficient concentration to increase production of said protein of interest relative to cells grown without said induction agent substantially decreasing cell viability.

The invention also comprises a perfusion system comprising, a cell line that expresses a protein of interest, a pre-sterilized disposable cell culture bioreactor, and culture media, wherein said culture media comprises an effective amount of an induction agent wherein cell viability does not substantially decrease and production of said protein of interest is increased relative to cells grown without said induction agent and wherein said pre-sterilized disposable cell culture bioreactor is partially filled with a gas comprising oxygen and said disposable cell culture bioreactor is agitated thereby agitating the liquid media in the bag. In one embodiment, at least about 80% to about 95% cell viability is maintained for at least 5 days in the presence of said induction agent. In another embodiment, said perfusion system comprises a filter wherein said filter concentrates said protein of interest in said pre-sterilized disposable cell culture bioreactor.

### BRIEF DESCRIPTION OF THE FIGURES

FIG. 1 is a graphical representation of the average daily yields using traditional perfusion methods and an intensified perfusion method (Raven Manufacturing Process or RaMP) utilizing the medium of the invention. The gray bars represent the average daily recombinant protein yields using traditional perfusion methods. The black bars represent the average daily recombinant protein yields using RaMP.

### DETAILED DESCRIPTION

As used herein, the term “induction agent” refers to an agent that increases the expression of mRNA and/or proteins in cells. The proteins can be naturally occurring in the cell or the product of recombinantly expressed proteins. Examples of induction agents include, but not limited to, members of the alkanoic acid family, for example sodium butyrate, sodium propionate, vanadate, and sodium orthovanadate.

As used herein, the terms “expression” or “expresses” refer to transcription and translation occurring within a host cell. The level of expression of a product gene in a host cell may be determined on the basis of either the amount of corresponding mRNA that is present in the cell or the amount of the protein encoded by the product gene that is produced by the cell. For example, mRNA transcribed from a product gene may be quantitated by northern hybridization and/or quantitative RT-PCR or any other suitable method known. Proteins quantities may be determined using immunoassays, ligand binding assays, enzymatic assays or any other protein quantitation method known in the art or developed in the future.

As used herein, an “effective amount of an induction agent” is the amount of an induction agent present in the media which will increase expression of mRNA and/or protein in cells by a detectable amount when compared to the cells grown without an induction agent.

As used herein, the phrase “cell viability does not substantially decrease” means that cell viability does not decrease any more than about 0% to about 20%, or about 5% to about 20%, or about 10% to about 15%. In other words, using the methods disclosed herein, at least about 80% to about 95% cell viability is maintained for at least about 5 days.

As used herein, the terms “continuous cell culture” or “continuous culture” refers to a culture characterized by both a continuous inflow of a liquid nutrient feed and a continuous liquid outflow. The nutrient feed may, but need not, be a concentrated nutrient feed. Continuously supplying a nutrient solution at about the same rate that cells are washed out of the reactor by spent medium allows maintenance of a culture in a condition of stable multiplication and growth. In a type of bioreactor known as a chemostat, the cell culture is continuously fed fresh nutrient medium, and spent medium, cells and excreted cell product are continuously drawn off. Alternatively, a continuous culture may constitute a “perfusion culture,” in which case the liquid outflow contains culture medium that is substantially free of cells, or substantially lower cell concentration than that in the bioreactor. In a perfusion culture, cells can be retained by, for example, filtration, centrifugation, or sedimentation.

As used herein, the term “serum free medium” refers to a medium lacking natural animal proteins. The hormones, growth factors, transport proteins, attachment factors, peptide hormones and the like typically found in serum that are necessary for the survival or growth of particular cells in culture are typically added as defined supplements to the serum free media.

As used herein, the terms “cell culture medium” and “culture medium” refer to the liquid solution which is used to provide sufficient nutrients (e.g., vitamins, amino acids, essential nutrients, salts, and the like) and properties (e.g., osmolality, buffering) to maintain living cells (or living cells in a tissue) and support their growth. The medium can also comprise additional factors including selection agents and induction agents. Commercially available tissue culture medium is known to those skilled in the art. Also, a person with skill in the art can formulate a culture media with defined components. The present invention is not limited to the use of a particular type of formulation of culture media.

As used herein, cells that have been “genetically engineered” to express a specific protein(s) when recombinant nucleic acid sequences that allow expression of the protein(s) have been introduced into the cells using methods of “genetic engineering,” such as viral infection with a recombinant virus, transfection, transformation, or electroporation. See e.g. Kaufman et al. (1990), Meth. Enzymol. 185, 487 to 511; Current Protocols in Molecular Biology, Ausubel et al., eds. (Wiley & Sons, New York, 1988, and quarterly updates). For the purposes of the invention, the antibodies produced by a hybridoma cell line resulting from a cell fusion are not “recombinant polypeptides.” These would be considered a naturally occurring protein. The methods of “genetic engineering” also encompass numerous methods including, but not limited to, amplifying nucleic acids using polymerase chain reaction, assembling recombinant DNA molecules by cloning them in Escherichia coli, restriction enzyme digestion of nucleic acids, ligation of nucleic acids, and transfer of bases to the ends of nucleic acids, among numerous other methods that are well-known in the art. See e.g. Sambrook et al., Molecular Cloning: A Laboratory Manual, 2nd ed., vol. 1-3, Cold Spring Harbor Laboratory, 1989.

Methods and vectors for genetically engineering cells and/or cell lines to express a protein of interest are well known to those skilled in the art. Genetic engineering techniques include but are not limited to expression vectors, targeted homologous recombination and gene activation. Optionally, the proteins are expressed under the control of a heterologous control element such as, for example, a promoter that does not in nature direct the production of that polypeptide. For example, the promoter can be a strong viral promoter (e.g., CMV, SV40) that directs the expression of a mammalian polypeptide. The host cell may or may not normally produce the protein. For example, the host cell can be a CHO cell that has been genetically engineered to produce a protein, meaning that nucleic acid encoding the protein has been introduced into the CHO cell. Alternatively, the host cell can be a human cell that has been genetically engineered to produce increased levels of a human protein normally present only at very low levels (e.g., by replacing the endogenous promoter with a strong viral promoter).

As used herein, the term “immunoglobulin” refers to a protein consisting of one or more proteins or polypeptides substantially encoded by immunoglobulin genes.

As used herein, the terms “antibody” and “antibodies” and refer to monoclonal antibodies, multispecific antibodies, fully human antibodies, humanized antibodies, camelised antibodies, chimeric antibodies, CDR-grafted antibodies, single-chain Fvs (scFv), disulfide-linked Fvs (sdFv), Fab fragments, F (ab′) fragments, anti-idiotypic (anti-Id) antibodies (including, e.g., anti-Id antibodies to antibodies of the invention) and other recombinant antibodies known to one skilled in the art and epitope-binding fragments of any of the above. In particular, antibodies include immunoglobulin molecules and immunologically active fragments of immunoglobulin molecules, i.e., molecules that contain an antigen binding site, these fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)-Fc fusions and scFv-scFv-Fc fusions. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the terms “protein,” “peptide” and “polypeptide” are used interchangeably to denote an amino acid polymer or a set of two or more interacting or bound amino acid polymers.

The inventors have discovered processes and methods to introduce induction agents in a traditional perfusion fermentation system without substantial loss in cell viability. This new method utilizes traditional perfusion methods to increase cell biomass and retains cell viability in the presence of induction agents. The disclosed processes and methods can result in increased biomass resulting in higher production of proteins when compared to both traditional perfusion and fed-batch methods. Because of the increased viability of the cells in the disclosed methods, the length of a production run can be infinite.

Accordingly, the present invention provides a process for perfusion cell culture of cells comprising culturing said cells in a nutrient medium supplemented with an induction agent, characterized in that following the addition of the induction agent the culture cells do not drop in viability. The cells in the perfusion cell culture of the invention have a relatively high viability level. Viability is defined as percentage of cells in the culture that are living. Preferably, viability in the cell culture system of the invention is more than about 70%, more preferably more than about 80%, most preferably above 90%. These percentages can be derived from the number of cells that are viable in a culture media or can be the percent of viable cells when compared to cells before the introduction of induction agents. Methods to measure cell viability comprise trypan blue exclusion assay, direct cell visualization of cells, O₂ uptake rate, fluorescent dyes such as Guava and Sytox and lactase dehydrogenase (LDH) assay.

In one embodiment, the present invention comprises a process for producing a protein of interest in a perfusion system, comprising culturing a cell line that expresses said protein of interest in media comprising an effective amount of an induction agent, whereby cell viability does not substantially decrease and production of said protein of interest is increased relative to cells grown without said induction agent. In another embodiment, about 80% to about 95% cell viability is maintained for at least 5 days in the presence of said induction agent. In another embodiment, least about 85% cell viability is maintained for at least 5 days in the presence of said induction agent.

In another embodiment, a biomass of at least about 4 million to about 60 million viable cells per milliliter is achieved in the presence of said induction agent for at least 5 days. In another embodiment, a biomass of at least about 5 million viable cells is achieved in the presence of said induction agent for at least 5 days. In another embodiment, a biomass of at least about 10, about 15, about 20, about 25, about 30, about 35, about 40, about 45, about 50, about 55, or about 60 million viable cells per milliliter is achieved in the presence of said induction agent for at least 5 days.

Any suitable induction agent may be used in the methods and cell culture systems of the invention. In one embodiment, said induction agent is selected from the group consisting of members of the alkanoic acid family, or salt thereof, sodium propionate, vanadate, sodium orthovanadate, DMSO, DMF, DMA, TNF-α, phorbol 12-myristate 13-acetate, PMA, propionate, forskolin, dibutyryl cAMP, 2-aminopurine, adenine, adenosine, okadaic acid, and combinations of any of these agents. The invention also comprises the use of any yet to be described and/or discovered induction techniques which will reduce cell viability when introduced to a cell culture system. In another embodiment, said induction agent is sodium butyrate. In another embodiment, the concentration of sodium butyrate is about 0.01 mM to about 50 mM. In another embodiment, the concentration of sodium butyrate is about 0.10 mM to about 20 mM of sodium butyrate. In another embodiment, the concentration of sodium butyrate is about 0.3 to about 10 mM. In another embodiment, the concentration of sodium butyrate is about 0.5 to about 2.5 mM. In another embodiment, the concentration of sodium butyrate is selected from the group consisting of about 0.5 mM, about 1.0 mM, about 1.5 mM, about 2.0 mM, about 2.5 mM, about 3.0 mM, about 3.5 mM, about 4.0 mM, about 4.5 mM, about 5.0 mM, about 5.5 mM, about 6.0 mM, about 6.5 mM, about 7.0 mM, about 7.5 mM, about 8.0 mM, about 8.5 mM, about 9.0 mM, about 9.5 mM and about 10.0 mM.

In another embodiment of the invention, the induction agent is introduced to the continuous or perfusion system over a period of time. One of the advantages of the continuous and perfusion systems is that fresh media can be added continuously. This allows an induction agent to be added to the media slowly in order to increase the concentration of the induction agent at a specific rate and/or over a period of time. Without being bound to any particular theory, the inventors believe that gradually introducing induction agents may help cells in the bioreactor adapt to the presence of the induction agent. Depending on the induction agent, the agent's final concentration may vary. The gradual increase to the desired final concentration may take hours to days. In another embodiment, the addition of the induction agent to its final concentration in the bioreactor will take approximately about 1 to about 10 days. In another embodiment, the addition of the induction agent to its final concentration in the fermentation chamber will take approximately about 2 to about 7 days. In another embodiment, the addition of the induction agent to its final concentration in the bioreactor will take approximately about 3 to about 5 days. In another embodiment, the concentration of the induction agent is increased to a final concentration of over a period of at least 2 days. In another embodiment, the addition of the induction agent to its final concentration in the bioreactor will take approximately about 2, about 3, about 4, about 5, about 6, about 7, about 9, or about 10 days. In another embodiment, the concentration of sodium butyrate can be increased gradually to a final concentration of about 0.1 mM to about 10 mM over a period of at least 2 days. In another embodiment, the concentration of sodium butyrate can be increased gradually to a final concentration of about 0.5 mM to about 2.5 mM over a period of at least 2 days. In another embodiment, the concentration of sodium butyrate can be increased gradually about 1.0 mM, about 1.5 mM, about 2.0 mM, about 2.5 mM, about 3.0 mM, about 3.5 mM, about 4.0 mM or more within a 2 day to 10 day interval.

The increase of the induction agent over the above referenced period of time can be accomplished by adding said induction agent in at least two or more doses that are spread out over a period of time. In one embodiment, two doses are spread over a period of about 2, about 3, about 4, about 5, about 6, about 7, about 8, about 9, about 10 days or longer. In another embodiment, the doses are spread over a period spaced more than one day apart. As used herein the term “dose” refers to an infusion of induction agent into the media that increases the concentration of the induction agent in the bioreactor. Thus, once the agent is present at a specific concentration, the concentration will not substantially drop and another dose will increase the concentration of the induction agent in the bioreactor. In one embodiment, the final concentration of sodium butyrate is gradually increased to a final concentration of about 0.5 mM, about 1.0 mM, about 1.5 mM, about 2.0 mM, about 2.5 mM or more over a period of about 2, about 3, about 4, about 5, or about 6 days. In another embodiment, sodium butyrate is introduced into the bioreactor and gradually increased over about a 5 day period from about 0.5 mM to about 2.5 mM. The timing of the addition of the induction agent may vary depending on the induction agent and the particular cells employed. Different induction agents will require specific conditions that a person of skill in the art would readily take into consideration. In the case of sodium butyrate, the agent is added about 7 to about 10 days after inoculation of the cells into the bioreactors or when the cells reach a concentration of about 4 million to about 60 million cells per milliliter. Without being bound to a particular theory, the gradual increase of sodium butyrate, or other induction agent, into the bioreactor may help the cells adjust to the induction agent and may help reduce cell death.

This invention is of particular utility because it increases production of proteins, in part, because the culture in the bioreactor has an increased number of viable cells in the presence of an induction agent and because the production run can be extended, theoretically, indefinitely. In one embodiment, production of a protein or polypeptide of interest is increased when compared to cells in similar growth conditions without the induction agent, wherein the cells in either bioreactor do not significantly drop in viability. In one embodiment, production of said protein of interest is at least about 0.2 g/L/day to about 5.0 g/L/day (or greater) at a cell density of about 4 million cells/ml to about 60 million cells/ml in the presence of an induction agent (i.e. sodium butyrate). In another embodiment, production of said protein of interest is at least about 0.5 g/L/day to about 1.0 g/L/day at a cell density of about 5 million cells/ml to about 30 million cells/ml in the presence of an induction agent. In another embodiment, production of said protein of interest is at least about 0.6 g/L/day at a cell density of about 60 million cells/ml in the presence of an induction agent.

The methods of the invention allow the cells in the bioreactor to stay viable indefinitely at a concentration of cells up to about 60 million cells/ml or greater. In one embodiment, approximately 10 million to 60 million cells/ml are viable in the presence of an induction agent. In another embodiment, approximately 10 million to 60 million cells/ml are viable in the presence of an induction agent for about 5, about 10, about 15, about 20, about 25, or about 30 days or longer. In another embodiment, there is no substantial drop in cell viability in the presence of an induction agent for about 5, about 10, about 15, about 20, about 25, or about 30 days or longer. In another embodiment, there is at least 85% viable cells in the presence of an induction agent for about 5, about 10, about 15, about 20, about 25, or about 30 days or longer.

In another embodiment at least about 80% to about 95% cell viability is maintained for at least 5 days in the presence of said induction agent. In another embodiment at least about 80% to about 95% cell viability is maintained for at least 5 days in the presence of said induction agent. In yet another embodiment at least about 80% to about 95% cell viability is maintained for about 10, about 15, about 20, about 25, or about 30 days or longer. In another embodiment, there is at least 85% viable cells in the presence of an induction agent for about 5, about 10, about 15, about 20, about 25, or about 30 days or longer.

Cells used in the process or methods of the invention typically, but need not be, the product of recombinant DNA technology. Said cells can be prokaryotic, fungal, insect, amphibian, mammalian, or other animal cells, such as chickens and fish. Preferably, the cells are derived from vertebrate organisms, more preferably, derived from mammalian cells. Non-limiting examples of examples of mammalian cells are COS cells, baby hamster kidney cells, mouse L cells, LNCaP cells, Chinese hamster ovary (CHO) cells, human embryonic kidney (HEK) cells, African green monkey cells, CV1 cells, HeLa cells, MDCK cells, Vero, NS0, and Hep-2 cells. In one embodiment, said mammalian cell is a CHO cell. In another embodiment, said CHO cell expresses a heterologous protein. Examples of insect cells are, Spodoptera frugiperda (Sf) cells, e.g. Sf9, Sf21, Trichoplusia ni cells, e.g. High Five cells, and Drosophila S2 cells. Examples of fungi (including yeast) host cells are S. cerevisiae, Kluyveromyces lactis (K. lactis), species of Candida including C. albicans and C. glabrata, Aspergillus nidulans, Schizosaccharomycespombe (S. pombe), Pichia pastoris, and Yarrowia lipolytica. Xenopus laevis oocytes, or other cells of amphibian origin, may also be used. Prokaryotic host cells include bacterial cells, for example, E. coli, B. subtilis, and Salmonella.

In another embodiment, the protein of interest is a natural protein produced by the cell. This would comprise a protein from the cell that was not genetically engineered to express the protein. These would include naturally transformed cells or cells naturally infected with virus. In another embodiment, the protein of interest is a recombinant protein. Recombinant proteins are proteins produced by the process of genetic engineering. The term “genetic engineering” refers to a recombinant DNA or RNA method used to create a host cell that expresses a gene at elevated levels, at lowered levels, or a mutant form of the gene. In other words, the cell has been transfected, transformed or transduced with a recombinant polynucleotide molecule, and thereby altered to cause the cell to alter expression of a desired protein. Methods and vectors for genetically engineering cells and/or cell lines to express a protein of interest are well known to those skilled in the art; for example, various techniques are illustrated in Current Protocols in Molecular Biology, Ausubel et al., eds. (Wiley & Sons, New York, 1988, and quarterly updates) and Sambrook et al., Molecular Cloning: A Laboratory Manual (Cold Spring Laboratory Press, 1989). In one embodiment, said recombinant protein is a therapeutic protein.

Any recombinant protein produced in cell culture can be manufactured using the methods of this invention. By way of example without limitation, fusion proteins, cell receptors, currently marketed recombinant proteins, such as EPO, or any protein used for manufacture of diagnostics or for laboratory purposes, such as cytokines, hormones, antigens, antibodies, can be produced using the processes and methods of the invention. In one embodiment, the recombinant protein is an immunoglobulin. In another embodiment, said immunoglobulin is an antibody. In another embodiment, said recombinant protein is express from a mammalian cell. In another embodiment, said mammalian cell is a CHO cell.

The invention also encompasses methods of culturing a cell line that expresses a protein of interest in a perfusion system, comprising culturing said cell line in media comprising an effective amount of an induction agent, whereby cell viability does not substantially decrease and production of said protein of interest is increased relative to cells grown without said induction agent. In one embodiment, at least about 80% to about 95% cell viability is maintained for at least 5 days in the presence of said induction agent. In another embodiment, at least about 85% cell viability is maintained for at least 5 days in the presence of said induction agent. In another embodiment, a biomass of at least about 2 million to about 60 million viable cells per milliliter is achieved in the presence of said induction agent for at least 5 days. In another embodiment, said perfusion system comprises a filter wherein said filter concentrates said protein of interest in a cell bioreactor.

### Continuous Culture And Perfusion Systems

The present invention employs both continuous and perfusion culture systems. The continuous culture system is typically used to extend the growth phase of the culture cells over long periods of time by providing fresh medium to the cells while simultaneously removing spent medium and cells from the bioreactor. Such a culturing system serves to maintain optimal culturing conditions for certain host cell types and proteins of interest. In addition, continuously supplying a nutrient solution at about the same rate that cells are washed out of the bioreactor by spent medium allows maintenance of a culture in a condition of stable multiplication and growth. A continuous culture may constitute a “perfusion culture,” in which case the liquid outflow contains culture medium that is substantially free of cells, or substantially lower cell concentration than that in the bioreactor. In a perfusion culture, cells can be retained by, for example, filtration, ultrasonic filtration, centrifugation, or sedimentation.

In one embodiment, the spent media is removed and sent through a filtration system that prevents cells from being removed from the bioreactor. In another embodiment, said filtration system comprises a hollow fiber filter. In another embodiment, the cells are prevented from being removed from the bioreactor by a centrifugation step. In another embodiment, the cells are prevented from being removed from the bioreactor by an ultrasonic filtration step. In another embodiment, the cells are prevented from being removed from the bioreactor via a sedimentation system as described in U.S. Pat. No. 5,817,505, herein incorporated by reference in its entirety for all purposes.

One drawback to some perfusion systems is that, as the spent media is washed out, the protein and/or peptide of interest is also washed out. This problem is overcome by either storing the spent media and purifying the protein of interest from it after the production run is completed or purifying the protein of interest out of the media while the media is being flushed from the bioreactor. Another method is to attach a filter to trap the protein of interest in the bioreactor and, thus, said protein of interest will accumulate in the bioreactor from which it can then be recovered. In one embodiment of the present invention, the perfusion system comprises a filter, wherein said filter concentrates said protein of interest in the bioreactor. In another embodiment, the filter is a 30 K filter.

In yet another embodiment, the perfusion system comprises a hollow fiber filter that will retain cells, but not the desired product. The cells are recycled back into the bioreactor and the spent media containing the desired product is passed through a desired molecular weight cut-off filter. The filter will retain the desired product and recycle it back into the bioreactor, thus concentrating the product. Waste products not retained by the filter can be disposed or recycled.

One embodiment of the invention comprises a perfusion system comprising, a cell line that expresses a protein of interest and culture media, wherein said culture media comprises an induction agent in sufficient concentration to increase production of said protein of interest relative to cells grown without said induction agent substantially decreasing cell viability. In another embodiment, at least about 80% to about 95% cell viability is maintained for at least 5 days in the presence of said induction agent. In another embodiment, at least about 85% cell viability is maintained for at least 5 days in the presence of said induction agent. In another embodiment, a biomass of at least about 4 million to about 60 million viable cells per milliliter is achieved in the presence of said induction agent for at least 5 days.

In another embodiment, said perfusion system employs at least one induction agent selected from the group consisting of members of the alkanoic acid family, or salt thereof. In another embodiment, said induction agent is sodium butyrate. In another embodiment, the concentration of sodium butyrate is from about 0.01 mM to about 50 mM. In another embodiment, the concentration of sodium butyrate is from about 0.1 mM to about 20 mM. In another embodiment, the concentration of sodium butyrate is from about 0.3 mM to about 10 mM. In another embodiment, the concentration of sodium butyrate is from about 0.5 mM to about 2.5 mM. In another embodiment, the concentration of sodium butyrate is increased to a final concentration over a period of at least 2 days. In another embodiment, said perfusion system comprises a filter wherein said filter concentrates said protein of interest in a fermentation chamber.

Any conventional bioreactor vessel can be used as the vessel for the purpose of this invention. The vessel may be made of materials such as stainless steel, glass, plastic, and/or ceramics, and may have a volume of from about 100 ml to 10,000 L or larger. In a preferred embodiment, the bioreactor is a disposable cell bioreactor. Disposable bioreactors are preferred over traditional bioreactors because they demonstrate reduced cross contamination between productions runs, are subject to less regulation by regulatory agencies (less SOPs for cleaning and setup), there is a faster turn around between production runs and less capital investment required for large scale operations.

Thus, one embodiment of the invention comprises a perfusion system comprising, a cell line that expresses a protein of interest, a disposable cell bioreactor, and culture medium, wherein said culture medium comprises an effective amount of an induction agent wherein cell viability does not substantially decrease and production of said protein of interest is increased relative to cells grown without said induction agent and wherein said disposable bioreactor. In another embodiment, said disposable bioreactor is pre-sterilized. In another embodiment, said disposable bioreactor is partially filled with a gas comprising oxygen and said bioreactor is agitated thereby agitating the liquid media in the disposable bioreactor. In another embodiment, the disposable bioreactor comprises a disposable plastic liner, for example, the plastic liners sold by Hyclone (Logan, Utah). In another embodiment, the disposable bioreactor comprises a pre-sterilized plastic bag, for example, the pre-sterilized plastic bags sold by Wave Biotech (Bridgewater, N.J.). In another embodiment, said pre-sterilized bag is partially filled with a gas comprising oxygen and said bag is rocked back and forth thereby inducing a wave motion into the liquid media in the bag.

In another embodiment of the invention, the disposable cell bioreactor of the perfusion system comprises a hollow fiber cell culture system, for example the FiberCell System sold by FiberCell Systems, Inc. (Frederick, Md.). Fresh media is perfused into one end the lumen of the fibers and the cells are grown on the outside of the fibers in the extracapillary space. The molecular weight cut off of the fibers allow for nutrients, waste products, induction agents, and other compounds to diffuse through, but not the cells or the desired product. The media containing waste products is then perfused out of the fibers through the other end.

In another embodiment, at least about 80% to about 95% cell viability is maintained for at least 5 days in the presence of said induction agent in said disposable cell bioreactor. In another embodiment, said perfusion system has at least about 85% cell viability for at least 5 days in the presence of said induction agent.

In another embodiment, said perfusion system has at least about 4 million to about 60 million viable cells per milliliter in the presence of said induction agent for at least 5 days. In another embodiment, a biomass of at least about 4 million viable cells per milliliter is achieved in the presence of said induction agent for at least 5 days in said disposable cell bioreactor. In another embodiment, a biomass of at least about 10, about 15, about 20, about 25, about 30, about 35, about 40, about 45, about 50, about 55, or about 60 million viable cells per milliliter is achieved in the presence of said induction agent for at least 5 days in said disposable cell bioreactor.

In another embodiment, said induction agent is selected from the group consisting of alkanoic acids, sodium propionate, vanadate, sodium orthovanadate, DMSO, DMF, DMA, TNF-α, phorbol 12-myristate 13-acetate, PMA, propionate, forskolin, dibutyryl cAMP, 2-aminopurine, adenine, adenosine, okadaic acid, and combinations thereof. In another embodiment, said induction agent is sodium butyrate.

In another embodiment, said disposable cell bioreactor comprises a filter wherein said filter concentrates said protein of interest. Depending on the size of the protein, any size filter may be optionally used in the culture system of the invention. In another embodiment, said filter has a 30K cutoff.

The rate at which the media is added and removed can affect the cell viability. Due to the nature of the induction agents, the media in-flow and out-flow rates may be vital. For example, the slower the rate, the more concentrated the protein of interest becomes and the less media will be required to put through down-stream purification. However, if perfusion is too slow, the cell viability/density tends to drop (because no new nutrients or not enough new nutrients are perfused into the bioreactor). Thus, in one embodiment of the invention, the cell culturing system of the invention permit an adjustable rate of perfusion such that cell viability is maintained at about 80%, about 85%, about 90% or about 95%. In another embodiment, said flow rate with the disclosed media is at about 0.5 to about 5.0 volumes/day. In another embodiment, said flow rate with the disclosed media is at about 0.8 to about 2.0 volumes/day. In another embodiment, said flow rate with the disclosed media (esp. RAV 12.1 basal media, see below) is at about 1.0 to about 1.5 volumes/day. This particular rate maintains high cell viability (over 85%) and high cell density (upwards of about 40 million to about 60 million cells/ml and higher) using the disclosed media with and without said induction agent.

The invention also encompasses methods of culturing a cell line using the systems of the invention. For instance in one embodiment, the invention comprises a method that expresses a protein of interest in a perfusion system utilizing a disposable cell bioreactor, comprising culturing said cell line in media comprising an effective amount of an induction agent wherein cell viability does not substantially decrease and production of said protein of interest is increased relative to cells grown without said induction agent. In another embodiment, said disposable bioreactor pre-sterilized. In another embodiment, said pre-sterilized disposable bioreactor is partially filled with a gas comprising oxygen and said bioreactor is agitated thereby agitating the liquid media in the disposable bioreactor. In another embodiment, the disposable bioreactor comprises a pre-sterilized plastic bag, for example, the pre-sterilized plastic bags sold by Wave Biotech (Bridgewater, N.J.). In another embodiment, said pre-sterilized bag is partially filled with a gas comprising oxygen and said bag is rocked back and forth thereby inducing a wave motion into the liquid media in the bag.

In another embodiment, at least about 80% to about 95% cell viability is maintained for at least 5 days in the presence of said induction agent in said disposable cell bioreactor. In another embodiment, said perfusion system has at least about 85% cell viability for at least 5 days in the presence of said induction agent. In yet another embodiment at least about 80% to about 95% cell viability is maintained for about 10, about 15, about 20, about 25, or about 30 days or longer. In another embodiment, there is at least 85% viable cells in the presence of an induction agent for about 5, about 10, about 15, about 20, about 25, or about 30 days or longer.

In another embodiment, said perfusion system has at least about 4 million to about 60 million viable cells per milliliter in the presence of said induction agent for at least 5 days. In another embodiment, a biomass of at least about 4 million viable cells per milliliter is achieved in the presence of said induction agent for at least 5 days in said disposable cell bioreactor. In another embodiment, a biomass of at least about 10, about 15, about 20, about 25, about 30, about 35, about 40, about 45, about 50, about 55, or about 60 million viable cells per milliliter is achieved in the presence of said induction agent for at least 5 days in said disposable cell bioreactor.

In another embodiment, said induction agent is selected from the group consisting of Alkanoic acids, sodium propionate, vanadate, sodium orthovanadate, DMSO, DMF, DMA, TNF-α, phorbol 12-myristate 13-acetate, PMA, propionate, forskolin, dibutyryl cAMP, 2-aminopurine, adenine, adenosine, okadaic acid, and combinations thereof. In another embodiment, said induction agent is sodium butyrate.

In another embodiment, said disposable cell bioreactor comprises a filter wherein said filter concentrates said protein of interest. In another embodiment, said filter has a 30K cutoff.

### Media Formulations And Culturing Conditions

Tissue culture medium is defined, for purposes of the invention, as a medium suitable for growth of cells, preferably mammalian cells, in in vitro cell culture. Typically, tissue culture medium contains a buffer, salts, energy source, amino acids, vitamins and trace essential elements. In addition, the medium can oftentimes require additional components such as growth factors, lipids, and/or other serum components (e.g., transferrin). The methods of the present invention are performed in a media formulation suitable for the particular cell line being cultured. Commercially available media such as Ham's F10 (Sigma), Minimal Essential Medium (MEM, Sigma), RPMI-1640 (Sigma), and Dulbecco's Modified Eagle's Medium (DMEM, Sigma) are exemplary nutrient solutions. In addition, any of the media described in Ham and Wallace, (1979) Meth. Enz., 58:44; Barnes and Sato, (1980) Anal. Biochem., 102:255; U.S. Pat. Nos. 4,767,704; 4,657,866; 4,927,762; 5,122,469 or 4,560,655; International Publication Nos. WO 90/03430; and WO 87/00195; the disclosures of all of which are incorporated herein by reference, may be used as the culture medium. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements (defined as inorganic compounds usually present at final concentrations in the micromolar range), vitamins (such as riboflavin, vitamin B-12), lipids (such as linoleic or other fatty acids) and their suitable carriers, selection molecules (methotrexate) and glucose or an equivalent energy source. Any other necessary supplements may also be included at appropriate concentrations that would be known to those skilled in the art.

In a particular embodiment, the mammalian host cell is a CHO cell (and any derivative thereof, e.g. CHO DUX (DHFR-), CHO K1, CHO DG44, CHO DP12). A suitable medium for CHO cells usually contains a basal medium component such as a DMEM/HAM F-12 based formulation (for composition of DMEM and HAM F12 media and especially serum free media, see culture media formulations in American Type Culture Collection Catalogue of Cell Lines and Hybridomas, Sixth Edition, 1988, pages 346-349) with modified concentrations of some components such as amino acids, salts, sugar, and vitamins, and optionally containing glycine, hypoxanthine, and thymidine; recombinant human insulin, Gentamycin, trace elements, hydrolyzed peptone, such as Soy-Peptone (Hy Soy Peptone), Protease Peptone 2 and 3, Primatone HS or Primatone RL (Difco, USA; Sheffield, England), or the equivalent. A cell protective agent, such as Pluronic F68 or the equivalent Pluronic polyol may also be included. In one embodiment, the cell culture media is a serum free media. In another embodiment, the cell culture media comprises an induction agent.

In a preferred embodiment, the media is a media comprising the components in Table 1.

TABLE 1

| RAV 14.1 Basal Media | mg/l | | g/l |
|---|---|---|---|
| L-Alanine | 114.1 | | 0.1141 |
| L-Arginine*HCl | 2191 | | 2.191 |
| L-Asparagine*H2O | 150 | | 0.15 |
| L-Asparagine anhydrous | 25 | | 0.025 |
| L-Aspartic Acid | 163.1 | | 0.1631 |
| L-Cysteine*HCl*H2O | 175.6 | | 0.1756 |
| L-Cystine*2HCl | 91.2 | | 0.0912 |
| L-Glutamic Acid | 222.1 | | 0.2221 |
| Glycine | 105.1 | | 0.1051 |
| L-Histidine*HCl*H2O | 251.7 | | 0.2517 |
| L-Isoleucine | 144.4 | | 0.1444 |
| L-Leucine | 236.2 | | 0.2362 |
| L-Lysine*HCl | 511.4 | | 0.5114 |
| L-Methionine | 74.8 | | 0.0748 |
| L-Phenylalanine | 115.6 | | 0.1156 |
| L-Proline | 385.3 | | 0.3853 |
| L-Serine | 147.1 | | 0.1471 |
| L-Threonine | 214.1 | | 0.2141 |
| L-Tryptophan | 36.4 | | 0.0364 |
| L-Tyrosine*Na | 78.96 | | 0.07896 |
| L-Tyrosine*2Na | 104.0 | | 0.104 |
| L-Valine | 211.2 | | 0.2112 |
| D-Biotin | 0.0863 | | 0.0000863 |
| Choline Chloride | 143.6 | | 0.1436 |
| Folic Acid | 17.24 | | 0.01724 |
| myo-Inositol | 187.4 | | 0.1874 |
| Niacinamide | 4.366 | | 0.004366 |
| D-Pantothenic Acid | 6.38 | | 0.00638 |
| (hemicalcium) | | | |
| Pyridoxine*HCl | 4.617 | | 0.004617 |
| Riboflavin | 0.776 | | 0.000776 |
| Thiamine*HCl | 7.37 | | 0.00737 |
| Vitamin B-12 | 1.373 | | 0.001373 |
| glucose | 8000 | | 8 |
| glutamine | 1600 | | 1.6 |
| NaCl | 4900 | | 4.9 |
| MgSO4 | 97.67 | | 0.09767 |
| CaCl (anhydrous) | 165 | | 0.165 |
| KCl | 330 | | 0.33 |
| Ammonium Metavanadate | 0.0117 | | 0.0000117 |
| Cupric sulfate-5H₂O | 0.25 | | 0.000025 |
| Ferrous sulfate*7H2O | 8.34 | | 0.00834 |
| Manganese sulfate | 0.00151 | | 0.00000151 |
| Magnesium sulfate | 97.67 | | 0.09767 |
| Molybdic Acid*4H2O | 0.124 | | 0.000124 |
| (ammonium) | | | |
| Potassium Nitrate (KNO₃) | 0.076 | | 0.000076 |
| Sodium Phosphate | 1250 | | 0.125 |
| (NaH₂PO₄—H₂O) | | | |
| Sodium Selenite | 0.0343 | | 0.0000343 |
| Zinc Sulfate*7H2O | 8.63 | | 0.00863 |
| NaHCO3 | 2100 | | 2.1 |
| Hepes | 15 | mM | |
| OTHERS | | | |
| Linoleic Acid | 2.841 | | 0.002841 |
| Putrescine-2HCl | 1.61 | | 0.00161 |
| Pyruvic Acid-Na | 1210 | | 1.21 |
| Thioctic Acid | 2.06 | | 0.00206 |
| pH | 7.20 | | |
| Osmolality (mOsmo) | 320-350 | | |

The media formulation described above (called RAV 14.1 Basal Media or RBM 14.1) has been shown to be optimal for perfusion systems. This formulation supports growth of cells in the perfusion systems of the invention, up to about 60 million cells/ml. In addition, this formulation maintains cell viability in the presence of induction agents. Thus, in one embodiment of the invention, RAV 14.1 Basal Media comprises an induction agent. In another embodiment, RAV 14.1 Basal Media can maintain least about 80% to about 95% cell viability for at least 5 days in the presence of an induction agent. In another embodiment, said media can maintain cells at least about 85% cell viability for at least 5 days in the presence of said induction agent. In another embodiment, said media can maintain cells at least about 4 million to about 60 million viable cells in the presence of said induction agent for at least 5 days. In another embodiment, said induction agent is sodium butyrate. In another embodiment, said induction agent is sodium butyrate. In another embodiment, the final concentration of sodium butyrate in said media is about 0.01 mM to about 50 mM. In another embodiment, final concentration of sodium butyrate in said media is about 0.1 mM to about 20 mM. In another embodiment, final concentration of sodium butyrate in said media is about 0.3 mM to about 10 mM. In another embodiment, final concentration of sodium butyrate in said media is about 0.5 mM to about 2.5 mM. RAV 14.1 Basal media can also be utilized with other cell culture systems known in the art (e.g. batch, fed batch, continuous) or developed in the future.

Another media formulation that has been shown to be optimal for perfusion systems is RAV12.1 Basal Media or RBM 12.1; see co-owned US Application 60/838,865, which is herein incorporated by reference in its entirety. This formulation supports growth of cells in the perfusion system of the invention, up to about 60 million cells/ml. In addition, this formulation maintains cell viability in the presence of induction agents, similar to RAV14.1.

After the addition of the induction agent, the osmolality of the media may need to be shifted. “Osmolality” is a measure of the osmotic pressure of dissolved solute particles in an aqueous solution. The solute particles include both ions and non-ionized molecules. Osmolality is expressed as the concentration of osmotically active particles (i.e., osmoles) dissolved in 1 kg of solution (1 mOsm/kg H2O at 38° C., it is equivalent to an osmotic pressure of 19 mm Hg). “Osmolality,” by contrast, refers to the number of solute particles dissolved in 1 liter of solution. Because with the addition of the induction agent, the cells produce more protein in general and the cells may start to swell and eventually lyse. Changing the osmolality counteracts the swelling effect. In one embodiment of the invention, the osmolality is shifted to counteract the effects of cell swelling and/or lysing.

In another embodiment, the osmolality of the RAV 14.1 Basal Media is shifted from about 320 to about 500 m Osmo. Solutes which can be added to the culture medium so as to increase the osmolality include proteins, peptides, amino acids, hydrolyzed animal proteins such as peptones, non-metabolized polymers, vitamins, ions, salts, sugars, metabolites, organic acids, lipids, and the like. The medium can be supplemented to maintain the osmolality within the appropriate margins according to whatever scheme is being used to maintain the cell culture. In another embodiment, the culture system is a perfusion culture system and the medium is supplemented with and without an induction agent.

The methods and processes of the invention are devised to enhance growth of cells in the growth phase of the cell culture and to increase production of a protein of interest. In the growth phase, cells are grown under conditions for a period of time that is maximized for growth. Culture conditions, such as temperature, pH, dissolved oxygen and the like, are customized for a particular cell line. Such conditions will be apparent to the ordinarily skilled artisan. Generally, the pH is adjusted to a level between about 6.5 and 7.5 using either an acid or a base. A suitable temperature range for culturing cells, such as CHO cells, is between about 30 to 38° C., preferably about 37° C. In addition, the suitable dissolved oxygen concentration is usually between 5-90% of air saturation.

With respect to the culture temperature, although it is possible to culture at a low temperature from the start of culturing, it is preferable to first culture at a temperature that enables growth (primary culturing temperature), and then after obtaining a sufficient number of cells, culturing at a lower temperature (secondary culturing temperature). The primary culturing temperature referred to here is preferably the temperature optimal for growth, usually at a temperature of 36-38° C., while a temperature of 37° C. is the most common. After the cells reach a sufficient number of cells the secondary culturing temperature is shifted below the primary culturing temperature preferably about 30-35° C. and most preferably about 30-33° C. Without being bound to a particular theory, the temperature shift may maintain high cell viability for a longer period of time, reduce death rate, reduce media consumption rate, reduce specific oxygen uptake and improve tolerance against shear stress. Thus, reduced temperature in may enhance the production of the protein of interest and/or enhance cell viability, esp. in the presence of induction agents.

In addition, the temperature lowering time (temperature shift time) is preferably the time at which a maximum biomass is reached for a particular cell culture system. The temperature shift time in perfusion culture is preferably the time at which cell density becomes sufficiently high. However, since the cell density that can be achieved in continuous culture varies according to the properties of the cell line used (suspendability, adhesion, etc.), various culture conditions (medium, pH, DO, stirring rate, shape of culture vessel, circulation rate, perfusion rate, etc.) and so forth, it cannot be limited within a narrow range. However, it is typically about 10⁶ to 10⁸ cells/mL. In one embodiment of the invention, after growing cells in the optimum temperature until the desired density is reached, the temperature is lowered at least about 2° C. in order to increase viability and production of the desired protein. In another embodiment, CHO cells are grown in at a temperature of 37° C. until the cells reach the desired concentration (about 0.5× to about 6.0×10⁷ cells/milliliter) and then the temperature is shifted down to about 27° C. to about 35° C. In another embodiment, said cells are grown at a reduced temperature after reaching a cell concentration of about 4 million to about 60 million viable cells per milliliter. In another embodiment, said cells are grown at a reduced temperature after reaching a cell concentration of about 20 million viable cells per milliliter. In another embodiment, as the temperature is shifted down the osmolality is shift up. In another embodiment, the temperature shift, osmolality shift and introduction of the induction agent occurs simultaneously. In another embodiment, the temperature shift, osmolality shift and introduction of an induction agent occurs consecutively in any desired order.

The protein of interest is then purified, or partially purified, from such the media using known processes. By “partially purified” means that some fractionation procedure, or procedures, have been carried out, but that more polypeptide species (at least 10%) than the desired polypeptide is present. By “purified” is meant that the polypeptide is essentially homogeneous, i.e., less than 1% contaminating polypeptides are present. Fractionation procedures can include but are not limited to one or more steps of filtration, centrifugation, precipitation, phase separation, affinity purification, gel filtration, ion exchange chromatography, hydrophobic interaction chromatography (HIC; using such resins as phenyl ether, butyl ether, or propyl ether), HPLC, or some combination of above.

The invention also optionally encompasses further formulating the proteins of interest. By the term “formulating” is meant that the proteins can be buffer exchanged, sterilized, bulk-packaged and/or packaged for a final user. For purposes of the invention, the term “sterile bulk form” means that a formulation is free, or essentially free, of microbial contamination (to such an extent as is acceptable for food and/or drug purposes), and is of defined composition and concentration. The term “sterile unit dose form” means a form that is appropriate for the customer and/or patient administration or consumption. Such compositions can comprise an effective amount of the polypeptide, in combination with other components such as a physiologically acceptable diluent, carrier, or excipient. The term “physiologically acceptable” means a non-toxic material that does not interfere with the effectiveness of the biological activity of the active ingredient(s).

This invention is further illustrated by the following examples which should not be construed as limiting. The contents of all references, patents and published patent applications cited throughout this application, as well as the Figures are incorporated herein by reference.

### EXAMPLES

### Example 1

### General Materials And Methods

The production cell line used for all experiments is a CHO (Chinese Hamster Ovary) cell-derived cell line transfected with the desired construct to produce a recombinant IgG1 protein. For all of the experiments, the cell line was grown in the RBM12.1 media under selective pressure (100 μg/ml Hygromycin B and 500 nM methotrexate). The cells were seeded in at a density of 4×10⁵ to 1×10⁶ cells per milliliter, depending on bioreactor size. pH was kept at 7.1±0.05 and temperature was kept at 37° C.±0.5. The perfusion rate for both traditional perfusion and the methods of the invention (RaMP) was 1.0 to 2.0 volumes/day. The cell retention devices used were the hollow fiber system and/or the cell settler system. Because of the high density of cells achieved utilizing RaMP (upwards of 40 to 60 million cells/ml), the hollow fiber system proved to be problematic due to clogging of the fibers. The cell settler system (retention by gravity) seem to overcome this problem. Sodium butyrate was introduced into the bioreactor after the cells reached the desired cell density (around 20 million cells/ml). Once sodium butyrate was introduced into the perfusion system, the cells were kept at that sodium butyrate condition until the next higher dose. This gradual ramping of sodium butyrate concentration was continued until the desired final concentration of sodium butyrate was reached. Once the desired sodium butyrate concentration is reached, the cells are grown in media in media containing the desired sodium butyrate concentration for the rest of the production run.

### Example 2

### Comparison of Cell Viability Using Traditional Perfusion And RaMP Methods

The mammalian host cell line used is a Chinese hamster ovary (CHO) cell line. This cell line has been transfected with the light and heavy chains of a chimeric IgG1 monoclonal antibody. The cells were grown in RBM12.1 medium under selective pressure (100 μg/ml Hygromycin B and 500 nM methotrexate (MTX). The medium used for this example contained the components in Table 1. Additional factors such as 10 μg/ml recombinant human insulin and 0.1% F68 are also added to the medium.

While a variety of commonly used cell culture or production media may be used in the practice of this invention, presently preferred embodiments use serum-free cell culture media formulated for recombinant protein production. Such media may be found in co-owned Application 60/838,865, which is herein incorporated by reference in its entirety.

During the growth period, the cultures were controlled at pH 7.1±0.05 by the use of CO₂ gas (acid) and/or Na₂CO₃ (base). Temperature was controlled at 37°±0.5 Celsius. Oxygen level was maintained at about 100% air saturation. For the initial growth period, CO₂ level was maintained at 5%. When the cell density reached 5×1 ⁶ cells/ml, CO₂ gas to the culture was turned off. The osmolality of the culture was maintained between 325 to 400 mOsm with minimal osmolality drift.

In a typical production schedule, 5×10⁵ to 1×10⁶ cells were inoculated into the cell culture bioreactor. 1 to 1.5 volumes of media was perfused daily. Cell density and viability was monitored by pulling a sample from the bioreactor and counted using trypan blue staining for live/dead cells.

Ten days after inoculation, the production period was started with the gradual addition of sodium butyrate (Na butyrate) into the RaMP cultures. 1.5 volume of RBM12.1 medium with 1 mM Na butyrate was perfused into the biorector on day 1 of the production period. This concentration of Na butyrate was kept in the bioreactor using the RaMP method for the duration of the production period. For cells in the traditional perfusion method, RBM12.1 media without Na butyrate was perfused into the bioreactor at the same perfusion rate as used in the RaMP method. Cell viability was monitored daily. The results showed comparable cell viability between the cells grown in the traditional perfusion method as compared to cells grown in the RaMP method. The cells in the RaMP method sustained high cell viability even with the addition of Na butyrate. The results over the entire production period are summarized in Table 2 below.

TABLE 2
Cell Viability Comparison: Traditional Perfusion
vs. RaMP

| | % Viable Cells | |
|---|---|---|
| | Perfusion | % Viable Cells |
| Day | (Traditional) | RaMP |
| 10 | 96.8 | 97 |
| 11 | 97.8 | 97.6 |
| 12 | 97.2 | 96.4 |
| 13 | 96.2 | 97.1 |
| 14 | 96.7 | 95.7 |
| 15 | 96.3 | 96.1 |
| 16 | 96.7 | 97.2 |
| 17 | 95.8 | 96.4 |
| 18 | 96.1 | 97 |
| 19 | 95.7 | 96.9 |
| 20 | 95.6 | 95.8 |

### Example 3

### Effects of Sodium Butyrate Addition On Cell Viability

In order to investigate if the method used to deliver sodium butyrate into the cell culture bioreactor would effect cell viability, two modes of delivery were tested: bolus introduction of the desired sodium butyrate concentration and the gradual “ramping” to the desired sodium butyrate concentration. The growth period conditions were identical to those described above.

On Day 9, sodium butyrate was introduced into the bioreactors. In the bolus condition, 2 mM sodium butyrate (final concentration) was added to the medium and perfused into the bioreactor. In the RaMP condition, sodium butyrate was introduced starting at 0.5 mM (final concentration) and gradually increased to the maximal concentration by Day 14. The maximal concentration of sodium butyrate was maintained for the rest of the production run. Cell viability was determined by pulling samples from each bioreactor and trypan blue staining for live/dead cells. After the bolus addition of 2 mM sodium butyrate, the cell viability started to decline (after day 9). The bioreactor for the bolus condition was stopped on day 19 after the viability dropped below 50%. In contrast, the gradual ramping of sodium butyrate from day 9 to day 14 maintained high cell viability (over 85%). The results of cell viability during the entire production run is summarized in Table 3 below.

TABLE 3
Effects of Mode of Sodium Butyrate Addition on Cell
Viability

| Days in Culture | % viable cells (bolus) | % viable cells (RaMP) |
|---|---|---|
| 1 | 97.6 | 96.4 |
| 2 | 97.8 | 95.7 |
| 3 | 97 | 97.5 |
| 4 | 97 | 99 |
| 5 | 94 | 98.2 |
| 6 | 94 | 97 |
| 7 | 89 | 98.6 |
| 8 | 94 | 98.5 |
| 9 | 89 | 97.4 |
| 10 | 76 | 97 |
| 11 | 73 | 97.6 |
| 12 | 76 | 96.4 |
| 13 | 72 | 97.1 |
| 14 | 72 | 95.7 |
| 15 | 84 | 96.7 |
| 16 | 79 | 97.2 |
| 17 | 76 | 96.4 |
| 18 | 80 | 97 |
| 19 | 44 | 96.9 |
| 20 | | 95.8 |
| 21 | | 95.4 |
| 22 | | 98.7 |
| 23 | | 90.6 |
| 24 | | 89.7 |
| 25 | | 88.8 |

Additional experiments using higher concentrations of sodium butyrate induction were also performed. The cells were seeded and grown similar to the conditions described above. On day 7, 0.5 f sodium butyrate was introduced into the perfusion medium. On day 10, the sodium butyrate concentration was increased to 1.0 mM. On day 12, the sodium butyrate concentration was increased to 2.0 mM. On day 14, the sodium butyrate concentration was increased to 4.0 mM and on day 17, the sodium butyrate concentration was increased to 8.0 mM. Throughout the experiment, cell viability was over 85% and the viable cell concentration reached over 4.8×10⁷ cells/ml. The results are summarized in Table 4 below.

TABLE 4
Effects of increasing sodium
butyrate on cell viability

| | Na butyrate | | |
|---|---|---|---|
| Day | concentration | | % viable cells |
| 4 | 0 | | 99.1 |
| 5 | 0 | | 98.1 |
| 6 | 0 | | 98.4 |
| 7 | 0.5 | mM | 97.3 |
| 8 | 0.5 | mM | 97.8 |
| 9 | 0.5 | mM | 97.8 |
| 10 | 1.0 | mM | 97.8 |
| 11 | 1.0 | mM | 96.6 |
| 12 | 2.0 | mM | 97.2 |
| 13 | 2.0 | mM | 95.9 |
| 14 | 4.0 | mM | 97 |
| 15 | 4.0 | mM | 96.7 |
| 16 | 4.0 | mM | 94.2 |
| 17 | 8.0 | mM | 94.7 |
| 18 | 8.0 | mM | 93.3 |
| 19 | 8.0 | mM | 91.5 |
| 20 | 8.0 | mM | 88.1 |

### Example 4

### Comparison of Average Yields of Recombinant Protein Per Day Using Traditional Perfusion And RaMP Method

Cells were seeded at 5×10⁵ cells/ml and were allowed to grow for 9 days in the bioreactor before induction with butyrate for the RaMP method. Conditions for this growth period are similar to those described in Example 1 above. On day 10, media containing 0.5 mM sodium butyrate was perfused into the bioreactor. On day 14, media containing 1.0 mM sodium butyrate was perfused into the bioreactor.

Cell viability was monitored daily throughout the production period. Over the entire production period, cell viability remained high and never dropped below 85% for both traditional perfusion and RaMP methods.

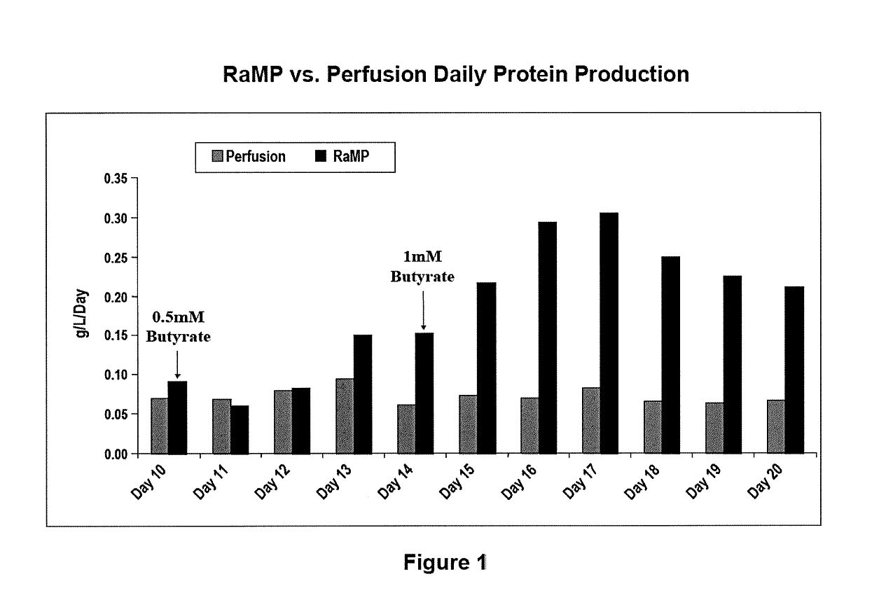

Samples of harvested media were taken from the daily harvest and frozen. At the end of the production run, the frozen daily samples were tested using an anti-IgG (recognizing only intact IgG) ELISA assay in order to determine the protein yields. FIG. 1 is a graphical representation of the average daily yield of recombinant protein from both traditional perfusion and RaMP methods.

### Example 5

### Average yields of recombinant protein per day using the RaMP method

Cells were seeded at 5×10⁵ cells/ml and were allowed to grow in the bioreactor for 6 days before the first harvest of recombinant protein on day 7. Conditions for this growth period are similar to those described in Example 1 above. Cells were grown in RBM12.1 media with a perfusion rate of 1 to 1.5 v/day. 1 mM sodium butyrate (final concentration) was added to perfusion medium and was perfused into the bioreactor on day 8. Cell viability was monitored and did not drop below 85% viability. 2 mM sodium butyrate (final concentration) was added to the perfusion media and was perfused into the bioreactor on day 11. Cell viability was monitored daily by pulling samples from the bioreactor and trypan blue live/dead staining. Over the production run, the cell density reached upwards of 4.5×10⁷ cells/ml. Using the RaMP method, the cell viability remained above 85% viable cells even with the sustained concentration of 2 mM sodium butyrate in the media. The average daily recombinant protein production reached over 0.6 g/L/day. The average daily recombinant protein production over the entire experiment is summarized in Table 5 below.

TABLE 5
Average yields per day using RaMP

| | | Average total |
|---|---|---|
| | Sodium Butyrate | production |
| Days in Culture | concentration | (mg/L/day) |
| 0-6 | 0 mM | — |
| 7 | 0 mM | 71 |
| 8 | 1 mM | 102 |
| 9 | 1 mM | 159 |
| 10 | 1 mM | 343 |
| 11 | 2 mM | 381 |
| 12 | 2 mM | 332 |
| 13 | 2 mM | 590 |
| 14 | 2 mM | 498 |
| 15 | 2 mM | 577 |
| 16 | 2 mM | 549 |
| 17 | 2 mM | 526 |
| 18 | 2 mM | 684 |

### Example 6

### Comparison of Average Daily Specific Productivity Using Traditional Perfusion, Fed-Batch And RaMP Methods

Average daily specific productivity was compared using traditional perfusion, fed-batch with sodium butyrate induction and RaMP processes. The same production cell line was used for all three methods. Culture conditions for all three methods were similar to those described in Example 1 above. Cells were inoculated at 5×10⁵ cells/ml and were allowed to grow to the desired concentration before induction with sodium butyrate. Fed-batch culture was induced with sodium butyrate as a bolus addition. Fed-batch specific productivity calculations were normalized and averaged for new protein production per day. Average daily specific productivity was calculated using production yields from a 10 day production run. Maximal sodium butyrate concentration was 2 mM for both fed-batch and RaMP processes. Average daily yields using the RaMP method was approximately 6 times more than fed-batch or traditional perfusion methods. The results are summarized in Table 6 below.

TABLE 6
Average Daily Specific Productivity for
Perfusion, Fed-batch and RaMP

| | |
|---|---|
| Perfusion | 0.07 g/L/day ± 0.01 |
| Fed-batch | 0.10 g/L/day ± 0.03 |
| RaMP | 0.64 g/L/day ± 0.07 |

The above experiments showed that the gradual ramping of sodium butyrate concentration to the desired maximal concentration preserved high cell viability (over 85% viable cells) as compared to a bolus addition of NaB. In one experiment, 0.5 mM NaB (final concentration) was introduced into the bioreactor by perfusing RBM12.1 medium with 0.5 mM NaB into the bioreactor on day 7 of the production run. On day 10, the bioreactor was perfused with RBM 12.1 medium with 1.0 mM NaB. On day 13, the bioreactor was perfused with RBM12.1 medium with 1.5 mM NaB. The cell viability did not decrease with the perfusion of higher NaB concentrations. These results also show that the disclosed RaMP method has higher specific productivity of proteins from cells in culture then compared to traditional perfusion methods and fed-batch method.

### Example 7

### Isoelectric Focusing Analysis of Chimeric IgG Proteins Produced Using Traditional Perfusion, Fed-Batch And RaMP Methods

The pI profile of purified chimeric IgG protein produced using traditional perfusion, fed-batch, and RaMP methods were determined using the following protocol, although other standard protocols can also be applied. The chimeric IgG protein was purified from the harvested culture medium using a Protein A column. The pI profile of the samples was determined using the Novex IEF gel and buffer system (Invitrogen). Gel samples were prepared by dilution with 2× sample buffer or dilution first in 1×PBS without Ca²⁺ and Mg²⁺ (Gibco, Catalog #20012) followed by addition of 2× sample buffer. pI markers ((Biorad cat #161-0310 and Invitrogen cat #39212-01) were diluted with 2× sample buffer. Diluted Ultrapure water (Gibco cat #10977) was diluted with 2× sample buffer for blank wells. All chimeric IgG protein samples were loaded at 5 μg/well.

Running buffers were prepared according to manufacture's instructions. Cathode buffer (10×) was diluted with MilliQ water to 200 ml of 1× concentration and filtered through a 0.2 μm filter and held under vacuum to degas solution for approximately 15 minutes. Anode buffer (50×) was diluted to 600 ml of lx concentration buffer. When degassing of the cathode buffer was complete, the pH 3-10, 1 mm 10-well gel (Invitrogen, Catalog #EC6655A) was removed from its packaging and prepared in a Novex gel chamber (Xcell SureLock Mini-cell) for electrophoresis. 1× cathode buffer was added to the top chamber and the 1× anode buffer was added to the lower chamber. Samples of purified chimeric IgG protein were loaded into the wells.

The gel was run for 90 minutes at 100V, then 90 minutes at 200V and then finally for 1 hour at 500V. Following the run, the gel was removed from the cassette and transferred to a gel tray containing approximately 100 ml of 12% trichloroacetic acid (Sigma, Catalog #T8657) in water. The gel was rocked gently for 30 minutes and then the fixative solution was removed. The gel was then rinsed in water and stained using 50 ml GelCode (Pierce, Catalog #24592) overnight at room temperature. The gel was then destained using MilliQ water for several hours, changing the water as needed.

The results of the IEF gel showed that the chimeric IgG protein purified from traditional perfusion, fed-batch culture and RaMP methods running in several major identical bands from a pI of 7.5 to 8.5. This result is indicative of similar product quality of the chimeric IgG proteins regardless of the method of production.

### Example 8

### SEC-HPLC Analysis of Chimeric IgG Proteins Produced Using Traditional Perfusion, Fed-Batch And RaMP Methods

Size exclusion chromatography (SEC-HPLC) analysis of chimeric IgG proteins produced using traditional perfusion, fed-batch and RaMP methods were determined using the following protocol, although other standard protocols can also be applied. Purified chimeric IgG proteins produced from the above methods were injected directly (with no dilution) onto a BioSep-SEC-S3000, 7.8×300 mm column (Phenomenex, Catalog #OOH-2146-K0) fitted with a compatible guard column (Phenomenex Security Guard) and ran at 0.3 ml/minute for 45 minutes in 200 mM sodium phosphate, 0.005% sodium azide, pH 7.0. Peak detection was at A280 using an Agilent 1100 HPLC system including a binary pump, autosampler, thermostated column compartment and multi-wavelength detector. Triplicate injections of each sample were performed. Integration data was evaluated and manual integration was performed as required. Average percent (%) monomer, standard deviation and % CV were calculated and reported for each test sample. The average percent monomers for the chimeric IgG proteins produced by each method were similar, indicating that the product quality of the chimeric IgG were comparable regardless of the method of production. The results are summarized below in Table 7.

TABLE 7
SEC-HPLC Analysis of chimeric IgG proteins produced using
traditional perfusion, fed-batch and RaMP methods.

| Sample | Ave % monomer | Std dev | % CV |
|---|---|---|---|
| RaMP | 99.31 | 0.11 | 0.11 |
| Traditional Perfusion | 99.52 | 0.02 | 0.02 |
| Fed-batch | 99.10 | 0.02 | 0.02 |

1. A process for producing a protein of interest in a perfusion system, comprising culturing a cell line that expresses said protein of interest in media comprising an effective amount of an induction agent, whereby cell viability does not substantially decrease and production of said protein of interest is increased relative to cells grown without said induction agent.

2. The process of claim 1, wherein at least about 80% to about 95% cell viability is maintained for at least 5 days in the presence of said induction agent.

3. The process of claim 2, wherein at least about 85% cell viability is maintained for at least 5 days in the presence of said induction agent.

4. The process of claim 1, wherein a biomass of at least about 4 million to about 60 million viable cells per milliliter is achieved in the presence of said induction agent for at least 5 days.

5. The process of claim 4, wherein a biomass of at least about 5 million viable cells per milliliter is achieved in the presence of said induction agent for at least 5 days.

6. The process of claim 4, wherein a biomass of at least about 10, about 15, about 20, about 25, about 30, about 35, about 40, about 45, about 50, about 55, or about 60 million viable cells per milliliter is achieved in the presence of said induction agent for at least 5 days.

7. The process of claim 1, wherein said induction agent is selected from the group consisting of members of the alkanoic acid family, or salt thereof.

8. The process of claim 7, wherein said members of the alkanoic acid family, or salt thereof, is selected from the group consisting of sodium butyrate, sodium propionate, vanadate, and sodium orthovanadate.

9. The process of claim 8, wherein said induction agent is sodium butyrate.

10. The process of claim 9, wherein the concentration of sodium butyrate is about 0.01 mM to about 50 mM.

11. The process of claim 10, wherein the concentration of sodium butyrate is about 0.1 mM to about 20 mM.

12. The process of claim 11, wherein the concentration of sodium butyrate is about 0.3 mM to about 10 mM.

13. The process of claim 12, wherein the concentration of sodium butyrate is about 0.5 mM to about 2.5 mM.

14. The process of claim 13, wherein the concentration of sodium butyrate is increased to a final concentration of about 0.5 mM to about 2.5 mM over a period of at least 2 days.

15. The process of claim 14, wherein the concentration of sodium butyrate is increased to a final concentration of about 0.5 mM to about 2.5 mM in at least two doses.

16. The process of claim 15, wherein the concentration of sodium butyrate is increased to a final concentration of about 0.5 mM to about 2.5 mM in at least two doses spaced more than one day apart.

17. The process claim 13, wherein said final concentration of sodium butyrate is about 2.0 mM.

18. The process of claim 1, wherein said production of said protein of interest is at least about 0.2 g/L/day to about 2.0 g/L/day at a cell density of about 4 million cells/ml to about 60 million cells/ml.

19. The process of claim 1, wherein said protein is a recombinant protein.

20. The process of claim 19, wherein said recombinant protein is an immunoglobulin.

21. The process of claim 1, wherein said cell line is a mammalian cell line.

22. The process of claim 21, wherein said mammalian cell is a CHO cell.

23. The process of claim 22, wherein said CHO cell expresses a heterologous protein.

24. The process of claim 23, wherein said heterologous protein is an immunoglobulin.

25. The process of claim 1, wherein said perfusion system comprises a filter, wherein said filter concentrates said protein of interest in a cell culture bioreactor.

26. The process of claim 1, wherein said cells are grown at a temperature of about 37° C.

27. The process of claim 26, wherein said temperature is reduced after reaching a cell concentration of about 4 million to about 60 million viable cells per milliliter.

28. The process of claim 27, wherein said temperature is reduced to about 27° C. to about 35° C.

29. The process of claim 1, wherein said cells are grown in RAV 12.1 Basal Media.

30. The process of claim 29, wherein the osmolality of said media is increased after the addition of said induction agent.

31. The process of claim 30, wherein said osmolality is increased to about 350 mOsmo to about 450 mOsmo.

32. The process of claim 31, wherein a shift in temperature occurs simultaneously with a shift in osmolality.

33. The process of claim 31, wherein a shift in temperature occurs consecutively with a shift in osmolality.

34. The process of claim 1 wherein said media is perfused at about 1 to about 1.5 volumes/day.

35. The process of claim 34, wherein said perfusion rate maintains cell viability of at least 85% and a cell density of about 40 to about 60 million cells/ml.

36. A method of culturing a cell line that expresses a protein of interest in a perfusion system, comprising culturing said cell line in media comprising an effective amount of an induction agent, whereby cell viability does not substantially decrease and production of said protein of interest is increased relative to cells grown without said induction agent.

37. The method of claim 36, wherein at least about 80% to about 95% cell viability is maintained for at least 5 days in the presence of said induction agent.

38. The method of claim 37, wherein at least about 85% cell viability is maintained for at least 10 days in the presence of said induction agent.

39. The method of claim 36, wherein a biomass of at least about 2 million to about 150 million viable cells per milliliter is achieved in the presence of said induction agent for at least 5 days.

40. The method of claim 39, wherein said biomass is about 4 million to about 100 million viable cells per milliliter.

41. The method of claim 40, wherein said biomass is about 20 million to about 80 million viable cells per milliliter.

42. The method of claim 41, wherein said biomass is about 40 million to about 60 million viable cells per milliliter.

43. The method of claim 39, wherein a biomass of at least about 4 million viable cells per milliliter is achieved in the presence of said induction agent for at least 5 days.

44. The method of claim 39, wherein a biomass of at least about 10, about 15, about 20, about 25, about 30, about 35, about 40, about 45, about 50, about 55, or about 60 million viable cells per milliliter is achieved in the presence of said induction agent for at least 5 days.

45. The method of claim 36, wherein said induction agent is selected from the group consisting of members of the alkanoic acid family, or salt thereof.

46. The method of claim 45, wherein said induction agent is sodium butyrate.

47. The method of claim 46, wherein the concentration of sodium butyrate is from about 0.5 mM to about 2.0 mM.

48. The method of claim 47, wherein said final concentration of sodium butyrate is 2.0 mM.

49. The method of claim 46, wherein the concentration of sodium butyrate is increased to a final concentration of about 0.5 mM to about 50 mM over a period of at least 2 days.

50. The method of claim 36, wherein said production of said protein of interest is at least about 0.2 g/L/day to about 2.0 g/L/day at a cell density of about 4 million to about 60 million cells/ml.

51. The method of claim 36, wherein said protein is a recombinant protein.

52. The method of claim 51, wherein said recombinant protein is an immunoglobulin.

53. The method of claim 36, wherein said cell line is a mammalian cell line.

54. The method of claim 53, wherein said cell line mammalian cell is a CHO cell.

55. The method of claim 54, wherein said CHO cell expresses a heterologous protein.

56. The method of claim 55, wherein said heterologous protein is an immunoglobulin.

57. The method of claim 36, wherein said perfusion system comprises a filter wherein said filter concentrates said protein of interest in a cell bioreactor.

58. A method of culturing a cell line that expresses a protein of interest in a perfusion system utilizing a pre-sterilized disposable bioreactor, comprising culturing said cell line in media comprising an effective amount of an induction agent wherein cell viability does not substantially decrease and production of said protein of interest is increased relative to cells grown without said induction agent and wherein said pre-sterilized disposable bioreactor is partially filled with a gas comprising oxygen and said pre-sterilized disposable bioreactor is agitated thereby agitating the liquid media in the pre-sterilized disposable bioreactor.

59. The method of claim 58, wherein at least about 80% to about 95% cell viability is maintained for at least 5 days in the presence of said induction agent.

60. The method of claim 59, wherein at least about 85% cell viability is maintained for at least 5 days in the presence of said induction agent.

61. The method of claim 58, wherein a biomass of at least about 4 million to about 60 million per milliliter viable cells is achieved in the presence of said induction agent for at least 5 days.

62. The method of claim 61, wherein a biomass of at least about 4 million viable cells per milliliter is achieved in the presence of said induction agent for at least 5 days.

63. The method of claim 61, wherein a biomass of at least about 10, about 15, about 20, about 25, about 30, about 35, about 40, about 45, about 50, about 55, or about 60 million viable cells per milliliter is achieved in the presence of said induction agent for at least 5 days.

64. The method of claim 58, wherein said induction agent is selected from the group consisting of members of the alkanoic acid family, or salt thereof.

65. The method of claim 64 wherein said induction agent is sodium butyrate.

66. The method of claim 65, wherein the concentration of sodium butyrate is from about 0.5 mM to about 50 mM

67. The method of claim 66, wherein the concentration of sodium butyrate is increased to a final concentration over a period of at least 2 days.

68. The method of claim 58, wherein said perfusion system comprises a filter wherein said filter concentrates said protein of interest in said pre-sterilized cell culture bioreactor.

69. The method of claim 68, wherein said filter has a 30K cutoff.

70. A perfusion system comprising, a cell line that expresses a protein of interest and culture media, wherein said culture media comprises an induction agent in sufficient concentration to increase production of said protein of interest relative to cells grown without said induction agent substantially decreasing cell viability.

71. The perfusion system of claim 70, wherein at least about 80% to about 95% cell viability is maintained for at least 5 days in the presence of said induction agent.

72. The perfusion system of claim 71, wherein at least about 85% cell viability is maintained for at least 5 days in the presence of said induction agent.

73. The perfusion system of claim 70, wherein a biomass of at least about 4 million to about 60 million viable cells per milliliter is achieved in the presence of said induction agent for at least 5 days.

74. The perfusion system of claim 73, wherein a biomass of at least about 5 million viable cells per milliliter is achieved in the presence of said induction agent for at least 5 days.

75. The perfusion system of claim 73, wherein a biomass of at least about 10, about 15, about 20, about 25, about 30, about 35, about 40, about 45, about 50, about 55, or about 60 million viable cells per milliliter is achieved in the presence of said induction agent for at least 5 days.

76. The perfusion system of claim 70, wherein said induction agent is selected from the group consisting of members of the alkanoic acid family, or salt thereof

77. The perfusion system of claim 76, wherein said induction agent is sodium butyrate.

78. The perfusion system claim 77, wherein the concentration of sodium butyrate is from about 0.5 mM to about 50 mM

79. The perfusion system of claim 78, wherein the concentration of sodium butyrate is increased to a final concentration over a period of at least 2 days.

80. The perfusion system of claim 70, wherein said perfusion system comprises a filter wherein said filter concentrates said protein of interest in a fermentation chamber.

81. The perfusion system of claim 80, wherein said filter has a 30K cutoff.

82. A perfusion system comprising, a cell line that expresses a protein of interest, a pre-sterilized disposable cell culture bioreactor, and culture media, wherein said culture media comprises an effective amount of an induction agent wherein cell viability does not substantially decrease and production of said protein of interest is increased relative to cells grown without said induction agent and wherein said pre-sterilized disposable cell culture bioreactor is partially filled with a gas comprising oxygen and said disposable cell culture bioreactor is agitated thereby agitating the liquid media in the bag.

83. The perfusion system of claim 82, wherein at least about 80% to about 95% cell viability is maintained for at least 5 days in the presence of said induction agent.

84. The perfusion system of claim 83, wherein is at least about 85% cell viability is maintained for at least 5 days in the presence of said induction agent.

85. The perfusion system of claim 82, wherein a biomass of at least about 4 million to about 60 million viable cells per milliliter is achieved in the presence of said induction agent for at least 5 days.

86. The perfusion system of claim 85, wherein a biomass of at least about 5 million viable cells per milliliter is achieved in the presence of said induction agent for at least 5 days.

87. The perfusion system of claim 85, wherein a biomass of at least about 10, about 15, about 20, about 25, about 30, about 35, about 40, about 45, about 50, about 55, or about 60 million viable cells per milliliter is achieved in the presence of said induction agent for at least 5 days.

88. The perfusion system of claim 82, wherein said induction agent is selected from the group consisting of members of the alkanoic acid family, or salt thereof

89. The perfusion system of claim 88, wherein said induction agent is sodium butyrate.

90. The perfusion system claim 89, wherein the concentration of sodium butyrate is from about 0.5 mM to about 50 mM

91. The perfusion system of claim 90, wherein the concentration of sodium butyrate is increased to a final concentration over a period of at least 2 days.

92. The perfusion system of claim 82, wherein said perfusion system comprises a filter wherein said filter concentrates said protein of interest in said pre-sterilized disposable cell culture bioreactor.

93. The perfusion system of claim 92, wherein said filter has a 30K cutoff.
